(19) United States
(12) Patent Application Publication
(10) Pub. No.: US 2002/0151578 A1
(43) Pub. Date: Oct. 17, 2002

(54) Formulation based on lipoic acid, process for its production and the use of this formulation for oral administration of lipoic acid

(76) Inventors: Jorg Breitenbach, Mannheim (DE); Thomas Hantke, Mannheim (DE); Bernd Liepold, Mannheim (DE); Jorg Rosenberg, Ellerstadt (DE)

Correspondence Address: Herbert B. Keil, KEIL & WEINKAUF, 1101 Connecticut Ave., N.W., Washington, DC, 20036, US

(21) Appl. No.: 10/080,668

(22) Filed: Feb. 25, 2002

(30) Foreign Application Priority Data
Feb. 26, 2001 (DE) 101 09 257.1

(51) Int. Cl. A61K 31/385; A61K 9/20

(52) U.S. Cl. 514/440; 424/465

(57) ABSTRACT

A formulation based i) on lipoic acid or a physiologically acceptable salt thereof and, where appropriate, other active substances, and a formulation base having ii) a binder component and iii) where appropriate other physiologically acceptable excipients is described. Lipoic acid is in the form of a molecular dispersion in these formulations. An advantageous process for their preparation, in particular by melt extrusion, and the use of this formulation for oral administration of lipoic acid are likewise described.

Formulation based on lipoic acid, process for its production and the use of this formulation for oral administration of lipoic acid.

The present invention relates to formulations based on lipoic acid in molecular dispersion, a process for their production, in particular by melt extrusion, and the use of this formulation for oral administration of lipoic acid.

As coenzyme in the oxidative decarboxylation of α-keto acids, lipoic acid is present in virtually every cell in an organism. Antiinflammatory, analgesic and cytoprotective properties, as well as its antioxidant effect, make lipoic acid an active substance of interest for pharmacy, cosmetics, food science and adjacent areas.

Formulations of solid salts of lipoic acid are proposed in U.S. Pat. Nos. 5,990,152. 5,994,393 relates to another modification of lipoic acid. Useful lipoic acid analogues are indicated in WO 99/45922.

However, lipoic acid is sensitive to oxidation and is moreover prone to polymerization. In addition, lipoic acid, and especially its R enantiomer, has a low melting point, at about 50° C. These are disadvantages which lead to considerable problems in the processing of lipoic acid. Apart from the fundamental difficulty of being able to formulate solid dosage forms, a decomposition of lipoic acid and thus a reduction in the lipoic acid content is observed even during the production process especially at higher processing temperatures. In addition, many preparations do not display adequate storage stability.

It is an object of the present invention to indicate lipoic acid-containing dosage forms of excellent quality, that is to say in particular with good storage stability and defined active substance content, and this object is achieved by formulations which comprise lipoic acid or a salt thereof in a molecular dispersion.

The present invention therefore relates to solid formulations based

i) on lipoic acid or a physiologically acceptable salt thereof and, where appropriate, other active substances and a formulation base having

ii) a binder component; and

iii) where appropriate, other physiologically acceptable excipients,

wherein lipoic acid or a physiologically acceptable salt thereof is in the form of a molecular dispersion.

The term “formulation” means for the purposes of the present invention a mixture composed of components i), ii) and, where appropriate, iii).

The term “lipoic acid” refers according to the invention to 5-(1,2-dithiolan-3-yl)valeric acid, also called thioctic acid, of the formula I

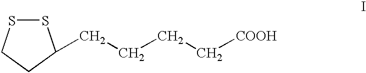

including the optical isomers covered by this formula, both as mixtures, e.g. racemates, and in pure form, e.g. R or S enantiomer. The preferred isomer is

(R)-5-(1,2-dithiolan-3-yl)valeric acid of the formula II

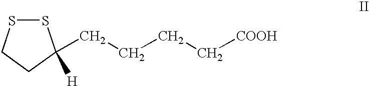

Lipoic acid with a (R) enantiomeric excess (ee) of at least 40% is preferred. The (R) enantiomeric excess is preferably at least 80%, in particular especially at least 98%.

The enantiomeric excess (ee) is derived from the following formula: ee[%]=(R−S)/(R+S)×100. R and S are the descriptors of the CIP system for the two enantiomers and represent the absolute configuration at the asymmetric C(5) atom. The enantiopure compound (ee=100%) is also referred to as homochiral compound.

The physiologically acceptable salts in the present case are preferably base addition salts.

The base addition salts include salts with inorganic bases, for example metal hydroxides or carbonates of alkali metals, alkaline earth metals or transition metals, or with organic bases, for example ammonia, basic amino acids such as arginine and lysine, amines, e.g. methylamine, dimethylamine, trimethylamine, triethylamine, ethylamine, diethylamine, ethylenediamine, ethanolamine, diethanolamine, 1-amino-2-propanol, 3-amino-1-propanol or hexamethylenetetraamine, saturated cyclic amines having 4 to 6 ring carbon atoms, such as piperidine, piperazine, pyrrolidine and morpholine, and other organic bases, for example N-methylglucamine, kreatine and tromethamine, and quaternary ammonium compounds such as tetramethylammonium and the like. Preferred salts with organic bases are formed with amino acids. Preferred salts with inorganic bases are formed with Na, K, Mg, Ca, Zn, Cr and Fe cations.

The active substance component i) of the formulations of the invention comprises lipoic acid. This may be present as free acid or as physiologically acceptable salt. Mixtures of these forms are possible, but will be considered only in certain cases. This part of the active substance component is for reasons of simplicity referred to hereinafter as lipoic acid content.

Besides the lipoic acid content, the formulation may comprise other active substances, in particular those with an action like that of lipoic acid, e.g. other antioxidants, vitamins, coenzymes and other active substances with nutritional significance, as well as expedient active substances of other types. One embodiment of the present invention comprises single-drug products which comprise as active substance component lipoic acid or a physiologically acceptable salt of lipoic acid.

The active substance component ordinarily constitutes 1 to 60% by weight, preferably 5 to 35% by weight and, in particular, 10 to 30% by weight of the formulation. Data in % by weight are based, unless indicated otherwise, on the total weight of the formulation.

The term “essentially” refers according to the invention usually to a percentage ratio of at least 90%, preferably of at least 95% and in particular of at least 98%.

The formulation base of formulations of the invention comprises physiologically acceptable excipients, namely at least one binder and, where appropriate, other physiologically acceptable excipients. Physiologically acceptable excipients are those known to be usable in the pharmaceutical and food technology sectors and adjacent areas, in particular those listed in relevant pharmacopeias (e.g. DAB, Ph. Eur., BP, NF), as well as other excipients whose properties do not impair a physiological use.

The term “molecular dispersion” is known to the skilled worker and describes essentially systems in which a substance, in the present case at least part and preferably the predominant part of the lipoic acid content, is homogeneously dispersed in a binder. In a molecular dispersion, the system is free of interfaces. The binder in this case usually forms a matrix which, according to the invention, is formed by the binder component or at least by a predominant part of the binder component.

The content of active substance crystals in a formulation of the invention is ordinarily below 12% and in particular below 5%. Statements about crystal contents relate to the total amount of the active substance(s), in particular the lipoic acid content.

A formulation of the invention which is essentially free of active substance crystals represents a particular embodiment of the present invention. The reduction in the crystal content is associated with an increase in the homogenization of the active substance in the matrix.

Molecular dispersion systems are, according to a particular embodiment, solid.

Formulations of the invention in which there are no crystalline contents for essentially any constituent (essentially amorphous or crystal-free formulations) represent a further particular embodiment of the present invention.

The state of such molecular dispersions can be investigated by known analytical methods, e.g. by differential scanning calorimetry (DSC) or wide-angle X-ray scattering measurements (WAXS measurements). Measurement of a molecular dispersion in DSC analysis lacks the, usually endothermic, melting peak occurring with the crystalline pure substance. Another possibility for identifying a molecular dispersion is the reduction in intensity and/or absence of typical X-ray diffraction signals in WAXS analysis.

The binder component of the formulations of the invention may also be understood as binder which at least in part forms a binder matrix, in particular a polymer matrix. Binders for the purpose of the invention are, in particular, solid meltable solvents. The binder matrix serves in particular to take up and, especially, to dissolve at least part of the active substance component, especially the lipoic acid content. To this extent the binder is also, in particular, a solvent. In relation to active substance which is in the form of a molecular dispersion and dissolved it is possible to speak of a solid solution.

According to a particular aspect, the invention relates to kinetically stabilized formulations.

The binder component is preferably at least partly soluble or swellable in aqueous media, expediently under the conditions of use, that is to say in particular physiological conditions.

Within the framework of the present description, aqueous media include water and mixtures of water and other components which comprise at least 50% by weight, preferably at least 70% by weight and in particular at least 90% by weight of water. Aqueous media include in particular body fluids such as fluids of the digestive tract, e.g. gastric juices, intestinal juices and saliva, blood; aqueous vehicles for use in pharmaceutical formulations in the drugs and food supplement sectors, e.g. vehicles which can be administered orally or parenterally, such as drinking water or water for injections.

Swelling means essentially a process in which the volume and/or shape of a solid body, for example of a solid formulation of the invention, change on exposure to liquids, vapors and gases. Swellable or soluble are, in particular, hydrophilic polymers which are able to accumulate water at least on the surface and/or take up water between the polymer chains, mainly by absorption. Limited swelling usually results in gel formation, which is why polymers capable of limited swelling and usable according to the invention can be selected from the polymers commonly known as gel formers. Unlimited swelling usually leads to the formation of solutions or colloidal solutions, which is why polymers capable of unlimited swelling and usable according to the invention can be selected from the polymers which form at least colloidal solutions in the particular aqueous medium. It is expidient to take into account, in particular in relation to body fluids, for example those of the gastrointestinal tract, that there may be local variations in the physiological conditions, especially the pH. If it is preferred, for example, that the active substance is taken up mainly in the duodenum, it may be advantageous for the binder component to be swellable under the conditions prevailing in the duodenum. In particular, it may be advantageous for only slight or, preferably, essentially no swelling to take place in the preceding sections of the gastrointestinal tract, especially in the stomach. However, it may be remarked at this point that such behavior of formulations of the invention can also be ensured with other means, in the case described above for example with coatings resistant to gastric juice or multilayer formulations in which usually inner layers containing active substance are exposed to swelling or dissolving only at the desired site.

Binder components technically preferred for the process are those which are melt-processable.

It is preferred for at least one binder of the binder component to be selected from:

synthetic polymers such as polyvinyllactams, in particular polyvinylpyrrolidone (PVP); copolymers of vinyllactams such as N-vinylpyrrolidone, N-vinylpiperidone and N-vinyl-ε-caprolactam, but especially N-vinylpyrrolidone, with (meth)acrylic acid and/or (meth)acrylic esters, such as long-chain (meth)acrylates, e.g. stearyl (meth)acrylate, dialkylaminoalkyl (meth)acrylates, which may be quaternized, and maleic anhydride, vinyl esters, especially vinyl acetate, vinylformamide, vinylsulfonic acid or quaternized vinylimidazole; copolymers of vinyl acetate and crotonic acid; partially hydrolyzed polyvinyl acetate; polyvinyl alcohol; (meth)acrylic resins such as poly(hydroxyalkyl (meth)acrylates), poly(meth)acrylates, acrylate copolymers, e.g. from alkyl acrylates with (meth)acrylic acid, and copolymers of dimethylaminoethyl acrylates and methacrylic esters (e.g. Eudragit types); polyalkylene glycols such as polypropylene glycols and polyethylene glycols, preferably with molecular weights above 1000, particularly preferably above 2 000 and very particularly preferably above 4 000 (e.g. polyethylene glycol 6 000); polyalkylene oxides such as polypropylene oxides and, in particular polyethylene oxides, preferably of high molecular weight, especially with weight average molecular weights of more than 100 000; copolymers of methyl methacrylate and acrylic acid; polyacrylamides, polyvinylformamide (where appropriate partially or completely hydrolyzed);

modified natural polymers, e.g. modified starches and modified celluloses, such as cellulose esters and, preferably cellulose ethers, e.g. methylcellulose and ethylcellulose, hydroxyalkylcelluloses, in particular hydroxypropylcellulose, hydroxyalkylalkylcelluloses, in particular hydroxypropylmethylcellulose or hydroxypropylethylcellulose, cellulose phthalates, in particular cellulose acetate phthalate and hydroxypropylmethylcellulose phthalate; starch degradation products, in particular starch saccharification products, such as maltodextrin;

natural or predominantly natural polymers such as gelatin, polyhydroxyalkanoates, e.g. polyhydroxybutyric acid and polylactic acid, polyamino acids, e.g. polylysine, polyasparagine, polydioxanes and polypeptides, and mannans, especially galactomannans; and

nonpolymeric binders such as polyols, for example those described in WO 98/22094 and EP 0 435 450, in particular sugar alcohols such as maltitol, mannitol, sorbitol, cellobiitol, lactitol, xylitol, erythritol and isomalt (Palatinit).

of those aforementioned, the polymeric binders, in particular the modified natural polymers, especially modified starches and cellulose ethers, and in particular the synthetic polymers, especially polyvinylpyrrolidones and polyvinylpyrrolidone copolymers, are preferred.

It is particularly preferred for at least one binder of the binder component to be selected from polyvinylpyrrolidones, N-vinylpyrrolidone/vinyl acetate copolymers, hydroxyalkylcelluloses, hydroxyalkylalkylcelluloses, cellulose phthalates, polyalkylene glycols, (meth)acrylic resins: for example the polyvinylpyrrolidones having the proprietary name Kollidon® and weight average molecular weights of about 2 000 to about 1.5×10⁶, for example the polyvinylpyrrolidone having the proprietary name Kollidon® 17 PF and a weight average molecular weight of about 7 000 to about 11 000; N-vinylpyrrolidone/vinyl acetate copolymers, in particular with a N-vinylpyrrolidone:vinyl acetate ratio of from about 30:70 to about 70:30, for example the product having the proprietary name Kollidon® VA 64 and a N-vinylpyrrolidone:vinyl acetate ratio of about 60:40; hydroxyalkylcelluloses with 1 to 3 carbon atoms in the alkyl moiety, in particular hydroxypropylcellulose, for example the hydroxypropylcellulose having the proprietary name Klucel®; hydroxyalkylalkylcelluloses with 1 to 3 carbon atoms in the alkyl moieties, in particular hydroxypropylmethylcellulose (HPMC), for example the methylcellulose and methylcellulose derivative mixtures having the proprietary name Methocel® and containing ethyl, hydroxyethyl, hydroxypropyl and carboxymethyl ether groups; cellulose phthalates, especially hydroxypropylmethylcellulose phthalate; polyalkylene glycols with 2 and/or 3 carbon atoms in the alkylene moiety, especially polyethylene glycols, for example the polyethylene glycols having the proprietary name Lutrol® and weight average molecular weights of from about 2 000 up to about 20 000, and polypropylene glycols; copolymers based on dimethylaminoethyl methacrylate and methacrylic esters such as methyl methacrylate and butyl methacrylate, for example the acrylic resins having the proprietary name Eudragit® E and based on dimethylaminoethyl methacrylate, methyl and butyl (meth)acrylate with weight average molecular weights of about 150 000, copolymers with anionic characteristics based on methacrylic acid and methyl methacrylate, for example the acrylic resins having the proprietary names Eudragit® L and S and with weight average molecular weights of 250 000 to 135 000.

Very particular preference is given to the aforementioned polyvinylpyrrolidones, N-vinylpyrrolidone/vinyl acetate copolymers and cellulose derivatives, especially Kollidon® VA 64, low molecular weight hydroxypropylcellulose, e.g. Klucel®EF with weight average molecular weights of about 45 000 to about 70 000 or about 80 000, and hydroxypropylmethylcellulose, e.g. Methocel® E3, E5 and E7. Polyvinylpyrrolidone K30 is preferred for the food supplement sector.

The binder component of the formulations of the invention preferably comprises at least one of the binders described above, in particular at least one polymeric binder. It may comprise other binders of these types and/or of other types. The properties of the formulation of the invention can be altered by the nature of the chosen binder or the admixture of different binders. In particular, it is possible in this way to control the release of active substance. From this viewpoint, the binder component preferably comprises at least one binder which slows the release of active substance at acidic pH. Polymers particularly suitable for this purpose from those mentioned above are those known also to be used in coatings resistant to gastric juice. These include, in particular, certain polyacrylates such as the Eudragit E and S types, and certain cellulose derivatives such as cellulose acetate phthalate or HPMC.

In one embodiment of the present invention, the binder component consists of one of the binders described above. In another embodiment of the present invention, the binder component consists of a mixture of at least two of the binders described above.

Polymers which are advantageous for use as polymeric binder are those which have a K value (according to H. Fikentscher, Cellulose-Chemie 13 (1932), pp. 58-64 and 71-74) in the range between 10 and 100, in particular between 15 and 80.

In a preferred embodiment, the binder component has a glass transition temperature of more than 80° C., preferably of more than 90° C. and in particular of more than 100° C. In addition, the suitability of glass transition temperatures in this range is governed by the necessary melt-processability of the binder or binder-containing mixtures.

The content of the binder component in the formulation of the invention is ordinarily from 20 to 99% by weight, preferably 30 to 90% by weight and in particular 40 to 80% by weight.

For the purpose of forming molecular dispersions and, in particular, solid solutions by at least part of the active substance component in the binder component, the content of active substance component based on the binder component is advantageously from 1 to 50% by weight, preferably 10 to 40% by eight and in particular 20 to 30% by weight.

Formulations of the invention may, besides binder component, contain further physiologically acceptable excipients (excipient component iii). Such excipients may facilitate production of the formulation and/or modulate its properties. The nature and amount are advantageously chosen so that they do not impair development of the special properties of the formulations of the invention, in particular the solid solution, or contribute to destabilizing this system.

Excipients are usually conventional pharmaceutical excipients, for example,

fillers such as sugar alcohols, e.g. mannitol, sorbitol, xylitol and isomalt (cf. DE 195 36 394), starch saccharification products, talc, sucrose, lactose, cereal or corn starch, potato flour, polyvinyl alcohol, where present in particular in a concentration of 0.02 to 50, preferably 0.20 to 20, % by weight based on the total weight of the mixture;

lubricants, glidants and mold release agents such as magnesium, aluminum and calcium stearates, talc and silicones, and animal or vegetable fats, especially in hydrogenated form and those which are solid at room temperature. These fats preferably have a melting point of 30° C. or above. Technically preferred in relation to the melt extrusion process are—as described in DE 197 31 277—triglycerides of C₁₂, C₁₄, C₁₆ and C₁₈ fatty acids or—to improve the processing properties—lecithin, as described in connection with the extrusion of an isomalt-containing polymer/active substance melt in DE 195 36 394. It is also possible to use waxes such as carnauba wax. These fats and waxes ay advantageously be admixed alone or together with mono- and/or diglycerides or phosphatides, in particular lecithin. The mono- and diglycerides are preferably derived from the abovementioned fatty acid types. Where present, the total amount of excipients in the form of lubricants and mold release agents is preferably 0.1 to 10% by weight and, in particular, 0.1 to 1% by weight, based on the total weight of the mixture;

flow regulators, e.g. diatomaceous earths, especially the high-purity silicon dioxides having the proprietary name Aerosil®, where present in particular in an amount of 0.1 to 5% by weight based on the total weight of the mixture;

dyes such as azo dyes, organic or inorganic pigments or dyes of natural origin, with preference being given to inorganic pigments here present in a concentration of 0.001 to 10, preferably 0.5 to 3% by weight, based on the total weight of the mixture;

stabilizers such as antioxidants, light stabilizers, hydroperoxide destroyers, radical scavengers, stabilizers against microbial attack;

plasticizers, especially those described below.

It is also possible to add wetting agents, preservatives, disintegrants, adsorbents and mold release agents, and surfactants, especially anionic and nonionic, such as, for example, soaps and soap-like surfactants, alkyl sulfates and alkylsulfonates, salts of bile acids, alkoxylated fatty alcohols, alkoxylated alkylphenols, alkoxylated fatty acids and fatty acid glycerol esters, which may be alkoxylated, and solubilizers such as Cremophor (polyethoxylated castor oil), Gelucire, vitamin E TPGS and Tween (ethoxylated sorbitan fatty acid esters) (cf., for example, H. Sucker et al. Pharmazeutische Technologie, Thieme-Verlag, Stuttgart 1978).

Excipients for the purpose of the invention also mean substances for producing a solid solution with the active substance. Examples of these excipients are pentaerythritol and pentaerythritol tetraacetate, urea, phosphatides such as lecithin, polymers such as, for example, polyethylene oxides and polypropylene oxides and their block copolymers (poloxamers) and citric and succinic acids, bile acids, stearins and others as indicated, for example, by J. L. Ford, Pharm. Acta Helv. 61, (1986), pp. 69-88.

Also regarded as pharmaceutical excipients are additions of acids and bases to control the solubility of an active substance (see, for example, K. Thoma et al., Pharm. Ind. 51, (1989), pp. 98-101).

The addition of antioxidants is a particular aspect according to the invention. These can be selected in particular from amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, aurothioglucose, propylthiouracil and other thiols (e.g. thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated dosages (e.g. pmol to μmol/kg), also (metal) chelators (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, e.g. propyl gallate, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate from gum benzoin, rutic acid and derivatives thereof, butylated hydroxytoluene, butylated hydroxyanisole, nordihydroguaiaretic acid, trihydroxybutyrophenone, uric acid and derivatives thereof, mannose and derivatives thereof, sesamol, sesamolin, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives suitable according to the invention (salts, esters, ethers, sugars, nucleotides, nucleosides, peptides and lipids) of these active substances mentioned. The amount of antioxidant to be added depends in particular on the purpose. A particular aspect is the use of antioxidant as polymerization inhibitor, especially for reducing the polymerization of lipoic acid to oligomers. The amount is therefore preferably chosen so that the amount of polymerized lipoic acid in the formulation is reduced. It is advantageous for the formulation to be essentially free of polymerized lipoic acid.

According to a particularly advantageous aspect, it is necessary to add no or only a small amount of antioxidant and/or polymerization inhibitor to prevent or reduce oxidation and/or polymerization of lipoic acid in formulations of the invention.

Excipients in the sense of the invention are also vehicles specific for the dosage form, i.e. appropriate for a particular dosage form, in particular peroral and, especially, tablets and capsules, also low-melting or liquid excipients such as polyalkylene glycols of low molecular weight, in particular polyethylene glycol and/or polypropylene glycol with weight average molecular weights of less than 1 000, water or suitable aqueous systems.

It is also possible to add excipients such as masking flavors and odor-masking agents, in particular sweeteners and odorants.

Further particular embodiments concerning excipients are based on expert knowledge as described, for example, in Fiedler, H. B., Lexikon der Hilfsstoffe fur Pharmazie, Kosmetik, und angrenzende Gebiete, 4th edition, Aulendorf: ECV-Editio-Cantor-Verlag (1996).

The only requirement for the suitability of excipients is usually the compatibility with the active substances and excipients used. The excipients ought advantageously not to impair the formation of molecular dispersions.

The excipient component in solid formulations of the invention preferably comprises at least one of the excipients described above. It may comprise other excipients of these types and/or other types.

One embodiment of the present invention comprises formulation bases with excipient component iii). In this case, the content of the other physiologically acceptable excipients in the formulations of the invention can be up to 91% by weight, preferably up to 60% by weight and, in particular, up to 40% by weight.

A particular embodiment of the present invention comprises formulations which comprise

i) lipoic acid or a lipoic acid salt;

ii) at least one binder selected from polyvinylpyrrolidones, N-vinylpyrrolidone copolymers, in particular with vinyl acetate, cellulose derivatives, in particular hydroxypropylcelluloses or hydroxypropylmethylcelluloses, and modified starches;

iii)where appropriate other excipients, for example a flow regulator, e.g. highly disperse silica gel.

The formulations of the invention preferably contain less than 5% by weight and, in particular, less than 2% by weight of water. A particular embodiment is represented by essentially anhydrous formulations.

From the viewpoint of a formulation which can be administered orally, it is particularly preferred for at least part of the binder component to be designed such that the release of active substance at acidic pH is delayed.

The formulations of the invention have a solid consistency. The term “solid” has in this connection the meaning assigned in relevant pharmacopeias in connection with pharmaceutical preparations. In the wider sense, solid formulations of the invention also include those with a semisolid consistency, which may result in particular with high lipoic acid contents. By this are meant viscous or highly viscous compositions which can be molded at room temperature. The suitability of semisolid formulations for being expediently processed, according to the invention in particular by means of extrusion, is important.

The present invention also relates to the use of formulations of the invention as dosage form preferably for oral administration of lipoic acid or of a physiologically acceptable salt thereof.

Accordingly, formulations of the invention are mainly used in the physiological, in particular in the medical, cosmetic and food technology sectors for humans and animals. In this sense, the formulations are used as or in dosage forms, i.e. the formulations of the invention have expedient forms appropriate for physiological practise, if necessary together with other excipients.

Thus, the term “dosage form” refers to any dosage form for administration of active substances to an organism, preferably to mammals, in particular humans, agricultural or domestic animals.

Conventional dosage forms include, in particular, (in alphabetical sequence) capsules, emulsions and microemulsions, granules, pellets, powders, suspensions, suppositories, tablets, especially coated tablets.

Emulsions and microemulsions may be of the oil-in-water or water-in-oil type and contain the formulations of the invention as disperse or dispersing phase. These emulsions or microemulsions may be stabilized by the presence of emulsifiers known to be used for this purpose.

Granules consist of solid grains of formulations of the invention, each grain representing an agglomerate of powder particles. Granules are preferably intended for oral use as dosage form. The user can be offered single-dose preparations, for example granules packed in a small bag (sachet), a paper bag or a small bottle, or multidose preparations which require appropriate measuring. However, in many cases, such granules do not represent the actual dosage form, but are intermediates in the manufacture of particular dosage forms, for example tablet granules to be compressed to tablets, capsule granules to be packed into hard gelatin capsules, or instant granules or granules for oral suspension to be put in water before intake.

As capsules, the formulations of the invention are usually packed into a hard shell composed of two pieces fitted together or a soft, one-piece, closed shell, which may vary in shape and size. It is likewise possible for formulations of the invention to be encased or enveloped or embedded in a matrix in suitable polymers, that is to say microcapsules and microspherules. Hard and soft capsules consist mainly of gelatin, while the latter have a suitable content of plasticizing substances such as glycerol or sorbitol. Hard gelatin capsules are used to receive preparations of the invention which have a solid consistency, for example granules, powder or pellets. Soft gelatin capsules are particularly suitable for formulations with a semisolid consistency and, if required, also viscous liquid consistency.

Pellets are granules of formulations of the invention in the particle size range from about 0.5 to 2 mm in diameter. Both with a narrow particle size distribution, preferably from 0.8 to 1.2 mm, and with an essentially round shape, are preferred.

In semisolid preparations, formulations of the invention are taken up in a suitable vehicle. Appropriate bases are known to the pharmaceutical or food technologist.

Suppositories are solid preparations for rectal, vaginal or urethral administration. In order to be appropriate for the administration route, formulations of the invention in these drug forms are usually taken up in suitable vehicles, for example in fats which melt at body temperature, such as hard fat, macrogols, i.e. polyethylene glycols with molecular weights of 1 000 to 3 000 in various proportions, glycerol gelatin and the like.

Tablets are solid preparations in particular for oral use. The meaning of oral within the framework of the present invention is, in particular, that of the term “peroral”, i.e. tablets for absorption or action of the active substance in the gastrointestinal tract. Particular embodiments are coated tablets, layered tablets, laminated tablets, tablets with modified release of active substance, matrix tablets, effervescent tablets, chewable tablets or pills. The formulations of the invention usually comprise at least a part of the necessary tablet excipients, such as binders, fillers, glidants and lubricants, and disintegrants. Tablets of formulations of the invention may also if necessary comprise other suitable excipients. Mention should be made in this connection of excipients which assist tableting, for example lubricants and glidants, for example those mentioned above, with preference for magnesium stearate in particular for facilitating compaction.

Coated tablets additionally comprise suitable coating materials, for example film coating agents with coating aids, especially those mentioned below. Coated tablets include, in particular, sugar-coated tablets and film-coated tablets.

Powders are finely dispersed solids of formulations of the invention with particle sizes usually of less than 1 mm. The above statements about granules apply correspondingly.

Preference is given according to the invention to capsules packed with comminuted granules, powders or pellets of formulations of the invention, instant granules and granules for oral suspension composed of formulations of the invention with addition of masking flavors, and, in particular, tablets.

The dosage forms of the invention are usually packed in a suitable form. Pushout packs made of plastic and/or metal for solid dosage forms are frequently used.

The present invention also relates to a process for producing a formulation of the invention by mixing (blending) components i), ii) and, where appropriate, iii) to form a plastic mixture. Thus, to form the plastic mixture, at least two measures are necessary, on the one hand the mixing (blending) of the components forming the mixture, and on the other hand the plastication thereof, i.e. the conversion thereof into the plastic state. These measures may take place for one or more components or portions of components successively, intermeshingly, alternately or in another way. Accordingly, it is possible in principle for the conversion into the plastic state to take place concurrently during a mixing process, or for the mixture first to be mixed and then to be converted into the plastic state. A plurality of plastic mixtures differing in composition may be formed during a process and are mixed together and/or with other components or portions of components. For example, a premix of a portion of the components, e.g. excipient component and/or binder component, can be granulated to form a plastic mixture, and the granules can then be converted, with the addition of other components, e.g. the active substance component, into another plastic mixture whose composition may correspond to that of the formulation. It is also possible for all the components first to be combined and then either converted into the plastic state at the same time of the mixing or first mixed and then converted into the plastic state.

The formation of a plastic mixture can take place by melting or—with additional input of mechanical energy, e.g. by kneading, mixing or homogenizing—else below the melting point of the mixture. The plastic mixture is preferably formed at temperatures below 220° C. The formation of the plastic mixture usually does not take place by one or more components being converted into a paste or partially dissolved with liquids or solvents, but takes place mainly or exclusively by thermal or thermal/mechanical action on the component(s), i.e. by thermal plastication. The plastic mixture is preferably formed by extrusion, particularly preferably by melt extrusion. The plastication process steps can be carried out in a manner known per se, for example as described in EP-A-0 240 904, EP-A-0 337 256, EP-A-0358 108, WO 97/15290 and WO 97/15291. The contents of these publications and, in particular, the statements about melt extrusion present therein are incorporated herein by reference.

It should be possible to convert the binder component into a plastic state in the complete mixture of all the components in the range from 30 to 200° C., preferably 40 to 170° C. The glass transition temperature of the mixture should therefore be below 220° C., preferably below 180° C. If necessary, it is reduced by conventional, physiologically acceptable plasticizing excipients.

Examples of such plasticizers are:

organic, preferably involatile compounds, such as, for example, C₇-C₃₀-alkanols, ethylene glycol, propylene glycol, glycerol, trimethylolpropane, triethylene glycol, butandiols, pentanols such as pentaerythritol and hexanols, polyalkylene glycols, preferably having a molecular weight of from 200 to 1 000, such as, for example, polyethylene glycols, polypropylene glycols and polyethylene/propylene glycols, silicones, aromatic carboxylic esters (e.g. dialkyl phthalates, trimellitic esters, benzoic esters, terephthalic esters) or aliphatic dicarboxylic esters (e.g. dialkyl adipates, sebacic esters, azelaic esters, citric and tartaric esters), fatty acid esters such as glycerol mono-, di- or triacetate or sodium diethyl sulfosuccinate. The concentration of plasticizer is, where present, generally 0.5 to 30, preferably 0.5 to 10, % by weight based on the total weight of polymer and plasticizer.

The amount of plasticizer advantageously does not exceed 30% by weight based on the total weight of polymer and plasticizer so that—in the area of solid forms—storage-stable formulations and dosage forms showing no cold flow are formed.

Account must further be taken of the fact that lipoic acid also has plasticizing properties so that the glass transition temperature of a mixture falls as the lipoic acid content thereof increases.

The process of the invention can advantageously be carried out at temperatures below 200° C. and preferably below 170° C., but above room temperature (25° C.), preferably above 40° C. A preferred temperature range for the extrusion of formulations of the invention is 80 to 150° C. The process is carried out in particular in a temperature range extending 40° C., preferably 30° C., and particularly preferably 20° C., upward or downward from the softening point of the mixture of the components.

In certain cases it may be advantageous to add components or portions of components as solution or suspension in a solvent. Particularly expedient ones are low molecular weight volatile solvents, e.g. water, C₁-C₆-monoalcohols and ethers thereof, esters of C₁-C₆-monoalkanols with C₁-C₆-carboxylic acids, alkanes. Another solvent which can be used is liquid CO₂. Water-soluble active substances can be employed as aqueous solution or, optionally, be taken up in an aqueous solution or dispersion of the binder component or a portion thereof. Corresponding statements apply to active substances which are soluble in one of the solvents mentioned, if the liquid form of the components used is based on an organic solvent. The components to be employed according to the invention may contain small amounts of solvent, e.g. because of hygroscopicity, trapped solvent or water of crystallization. The total solvent content of the plastic mixture is preferably less than 15%, in particular less than 10%, and particularly preferably less than 5%. The plastic mixture is preferably formed without the addition of a solvent, i.e. in particular by solvent-free melt extrusion.

The components, i.e. active substance and/or binder and, where appropriate, other excipients, can first be mixed and then be converted into the plastic state and homogenized. This can be done by operating the apparatuses such as stirred vessels, agitators, solids mixers etc. alternately. Sensitive active substances can then be mixed in (homogenized), preferably in “intensive mixers” in plastic phase with very small residence times. The active substance(s) may be employed as such, i.e. in particular in solid form, or as solution, suspension or dispersion.

The plastication, melting and/or mixing takes place in an apparatus usual for this purpose. Extruders or heatable containers with agitator, e.g. kneaders (like those of the type mentioned hereinafter) are particularly suitable.

It is also possible to use as mixing apparatus those apparatuses which are employed for mixing in plastics technology. Suitable apparatuses are described, for example, in “Mischen beim Herstellen und Verarbeiten von Kunststoffen”, H. Pahl, VDI-Verlag, 1986. Particularly suitable mixing apparatuses are extruders and dynamic and static mixers, and stirred vessels, single-shaft stirrers with stripper mechanisms, especially paste mixers, multishaft stirrers, especially PDSM mixers, solids mixers and, preferably mixer/kneader reactors (e.g. ORP, CRP, AP, DTB from List or Reactotherm from Krauss-Maffei or Ko-Kneader from Buss), trough mixers or internal mixers or rotor/stator systems (e.g. Dispax from IKA).

The process steps of mixing and plastication, that is to say in particular the melting, can be carried out in the same apparatus or in two or more apparatuses operating separately from one another. The preparation of a premix can be carried out in one of the mixing apparatuses described above and normally used in particular for granulation. Such a premix can then be fed directly for example into an extruder, and then be extruded where appropriate with the addition of other components.

It is possible in the process of the invention to employ as extruders single screw machines, intermeshing screw machines or else multiscrew extruders, especially twin screw extruders, corotating or counter-rotating and, where appropriate, equipped with kneading disks. If it is necessary in the extrusion to evaporate a solvent, the extruders are generally equipped with an evaporating section. Examples of extruders which can be used are those of the ZSK series from Werner & Pfleiderer.

The mixing apparatus is charged continuously or batchwise, depending on its design, in a conventional way. Powdered components can be introduced in a free feed, e.g. via a weigh feeder. Plastic compositions can be fed in directly from an extruder or via a gear pump, which is particularly advantageous if the viscosities and pressures are high. Liquid media can be metered in by a suitable pump unit.

The mixture which has been obtained by mixing and converting the polymer component, the active substance component and, where appropriate, other excipients into the plastic state is pasty, of high viscosity or low viscosity (thermoplastic) and can therefore also be extruded. The glass transition temperature of the mixture is advantageously below the decomposition temperature of all the components present in the mixture.

The formulation of the invention is suitable as plastic mixture—where appropriate after cooling or solidification—in particular as extrudate, for all conventional processes for manufacturing conventional dosage forms, in particular drug forms.

The present invention also relates to a process for producing dosage forms based on formulations of the invention. Thus, where the formulation can be produced by the above process, and the formulation can be converted into the required dosage form where appropriate with the addition of other excipients. This can be done by using shaping process measures such as shaping the plastic mixture, in particular by extrusion or melt extrusion, and shaping the plastic mixture, in particular the extrudate—where appropriate after cooling or solidification—for example by granulation, grinding, compression, casting, injection molding, tableting under pressure, tableting under pressure with heat. It is also possible to convert a formulation into a desired dosage form by introducing it into suitable vehicles. It is thus also possible to process solid formulations into semisolid or liquid formulations through the addition of suitable vehicles.

A large number of, in particular, solid dosage forms can be manufactured in this way. For example, powders or granules can be produced by grinding or chopping the solidified or at least partly solidified plastic mixture, and can be either used directly for treatment or, where appropriate with addition of conventional excipients, further processed to the above dosage, in particular drug forms, especially to tablets.

Dosage forms are preferably shaped before solidification of the plastic mixture and result in a form which can be employed for treatment where appropriate after coating in a conventional way.

The shaping to the dosage form before solidification can take place in a variety of ways depending on the viscosity of the plastic mixture, for example by casting, injection molding, compression, nipping or calendering. This is done by conveying the plastic mixture described above in the process according to the invention to one or more shaping steps. The conveying can take place by pressing, pumping, e.g. with gear pumps, or, preferably, with an extruder.

The plastic mixture is particularly preferably formed in one or more, preferably one, extruder and conveyed by the latter or a downstream extruder to the shaping steps. It has proved to be advantageous in many cases to extrude on a downward incline and/or where appropriate provide a guide channel for transporting the extrudate, in order to ensure safe transport and prevent rupture of the extrudate.

It may also be advantageous, depending on the number and compatibility of the active substances to be employed, to employ multilayer extrudates, for example coextrudates, as described in WO 96/19963, in the process of the invention.

Multilayer solid dosage forms can be produced in particular by coextrusion, in which case a plurality of mixtures of one or more of the components described above are conveyed together into an extrusion die so that the required layer structure results. Different binders are preferably used for different layers.

Multilayer dosage forms preferably comprise two or three layers. They may be in open or closed form, in particular as open or closed multilayer tablets.

If the shaping takes place by coextrusion, the mixtures from the individual extruders or other units are fed into a common coextrusion die and extruded. The shape of the coextrusion dies depends on the required dosage form. Examples of suitable dies are those with a flat orifice, called slit dies, and dies with an annular orifice cross section. The design of the die depends on the formulation base used and, in particular, the binder component and the desired dosage form.

The first shaping step advantageously takes place when the extrudate emerges from the extruder through suitably shaped dies, draw plates or other orifices, for example through a breaker plate, a circular die or a slit die. This usually results in a continuous extrudate, preferably with a constant cross section, for example in the form of a ribbon or of a strand, preferably with a circular, oval, rounded or flat and broad cross section.

Suitable downstream shaping steps for extrudates are, for example, cold cut, that is to say the cutting or chopping of the extrudate after at least partial solidification, hot cut, that is to say the cutting or chopping of the extrudate while still in the plastic form, or pinching off the still plastic extrudate in a nip device. It is possible with hot or cold cut to obtain, for example, granules (hot or cold granulation) or pellets. Hot granulation usually leads to dosage forms (pellets) with a diameter of from 0.5 to 3 mm, while cold granulation normally leads to cylindrical products with a length to diameter ratio of from 1 to 10 and a diameter of from 0.5 to 10 mm. It is possible in this way to produce monolayer but also, on use of coextrusion, open or closed multilayer dosage forms, for example oblong tablets, pastilles and pellets. The dosage forms can be provided with a coating by conventional methods in a downstream process step. Suitable materials for film coatings are the polymers mentioned as polymeric binders, in particular polyacrylates such as the Eudragit® types, cellulose esters such as the hydroxypropylcellulose phthalates, and cellulose ethers such as ethylcellulose, hydroxypropylmethylcellulose or hydroxypropylcellulose, and gelatin. Further shaping steps may also follow, such as, for example, rounding off the pellets obtained by hot or cold cut using rounding-off devices as described in DE-A-196 29 753.

It is particularly preferred for all the shaping steps to be carried out on the still plastic mixture or still plastic extrudate. Besides hot cut, where appropriate with subsequent rounding off, a particularly suitable process is one in which the plastic mixture is shaped to the dosage form in a molding calender. This is done by conveying a still plastic mixture or a still plastic extrudate to a suitable molding calender. Suitable molding calenders usually have molding rolls and/or belts for the shaping, with at least one of the molding rolls and/or at least one of the belts having depressions to receive and shape the plastic mixture. It is preferred to use a molding calender with counter-rotating molding rolls, with at least one of the molding rolls having on its surface depressions to receive and shape the plastic mixture. Suitable molding calenders and devices containing molding rolls are generally disclosed for example in EP-A-0 240 904, EP-A-0 240 906 and WO 96/19962, and suitable belts and devices containing belts are generally disclosed for example in EP-A-0 358 105, which are expressly incorporated herein by reference.

The shaping of the still plastic mixture or still plastic extrudate preferably takes place at melt temperatures below 220° C., particularly preferably below 180° C. and very particularly preferably below 150° C., such as, for example, in the temperature ranges necessary to form the plastic mixture or at lower temperatures. If the shaping takes place at lower temperatures, it advantageously takes place at from 5 to 70° C., preferably 10 to 50° C. and particularly preferably 15 to 40° C. below the highest temperature reached on formation of the plastic mixture, but preferably above the solidification temperature of the plastic. mixture.

The production according to the invention of the formulations and preparation of the dosage forms can be carried out wholly or partly under sterile operating conditions, for example in cleanrooms and with use of sterilized equipment such as, for example, weighers, mixers, extruders and shaping machines, such as calenders, nip devices and choppers. It is possible either for the starting materials to be introduced into the process in sterilized form, where appropriate with the addition of suitable antibacterial and/or antiviral excipients, and/or for the process conditions, especially the temperature, to be chosen such that sterile formulations or dosage forms are obtained. The resulting sterile dosage forms can then be packaged directly, likewise under sterile conditions, for example by blister packing or sealing. The shaping and the packaging may also be carried out at the same time, in particular when the shaping of the plastic mixture by calendering is carried out by molding rolls. This is done by introducing, in addition to the plastic mixture, materials in the form of sheets between the melt and the molding roll in each case, whereby it is possible to achieve at the same time as the shaping of the plastic mixture to dosage forms an enveloping and/or a packaging of the dosage form, as described in WO-96/19963, which is incorporated herein by reference.

Preference is given to formulations and dosage forms obtainable by one of the processes described above.

Formulation of the invention, where appropriate as dosage form, and thus an effective amount of active substance, are administered to the individual to be treated, preferably a mammal, in particular a human, agricultural or domestic animal. Whether such a treatment is indicated and what form it is to take depends on the individual case and may be subject to medical assessment (diagnosis) which includes the signs, symptoms and/or dysfunctions which are present, the risks of developing certain signs, symptoms and/or dysfunctions, and other factors. The formulations of the invention are ordinarily administered together or alternately with other products in such a way that an individual to be treated receives a daily dose of about 1 mg to 5 g, preferably of about 10 mg to 1 g, of lipoic acid on oral administration, and of about 5 mg to 1 g of lipoic acid on parenteral administration.

The formulations and dosage forms of the invention are mainly used in pharmacy, cosmetics and food technology, for example in the pharmaceutical sector as antiinflammatory, analgesic and cytoprotective agents, in the cosmetics sector as agents with an antioxidant effect and in the food technology sector as supplements to human and animal foods, e.g. within the framework of preventive nutritional strategies.

The present invention is now to be illustrated, but not restricted, by the present example.

### BEISPIEL 1

20% by weight of lipoic acid racemate are mixed with 80% by weight of Kollidon® VA-64. This mixture is continuously metered at 1 kg/h into a twin screw extruder (screw diameter 18 mm; 6 extruder sections). Extrusion takes place with the screw rotating at 40-80 rpm. The screws comprise in addition to conveying screw elements also so-called kneading elements and short return conveying elements which ensure adequate plastication of the mixture. The temperatures of the extruder housing are 10° C. (housing directly behind the intake housing, which is cooled) to 120° C. (housing 5 and extruder head/die). The lipoic acid content is determined by HPLC to be 99.8% of the amount employed.

1. A solid formulation based
i) on lipoid acid or a physiologically acceptable salt thereof and, where appropriate, other active substances and a formulation base having
ii) a binder component; and
iii) where appropriate, other physiologically acceptable excipients,
wherein lipoid acid or a physiologically acceptable salt thereof is in the form of a molecular dispersion.

2. A formulation as claimed in claim 1, wherein at least one binder of the binder component is selected from polyvinylpyrrolidones, N-vinylpyrrolidone copolymers, cellulose derivatives and modified starches.

3. A formulation as claimed in claim 1, wherein the binder component has a glass transition temperature of more than 80° C., preferably of more than 90° and in particular more than 100°.

4. A formulation as claimed in claim 1, wherein the formulation comprises
i) 1 to 60% by weight, preferably 5 to 35% by weight and in particular 10 to 30% by weight of active substance component;
ii) 20 to 99% by weight, preferably 30 to 90% by weight and in particular 40 to 80% by weight, of binder component;
iii) 0 to 91% by weight, preferably 1 to 60% by weight and in particular 5 to 40% by weight, of other physiologically acceptable excipients;

5. A formulation as claimed in claim 1, wherein the content of active substance component relative to binder component is from 1 to 50% by weight, preferably 10 to 40% by weight and in particular 20 to 30% by weight.

6. A formulation as claimed in claim 1, comprising
I) lipoid acid or a physiologically acceptable salt thereof;
ii) at least one binder selected from polyvinylpyrrolidones, -vinylpyrrolidone/vinyl acetate copolymers, hydroxypropyl-cellulose, hydroxyproplymethylcelluloses and modified starches; and
iii) where appropriate other physiologically acceptable excipients, in particular a flow regulator, e.g. highly disperse silica gel.

7. A formulation as claimed in claim 1 by melt extrusion of a mixture comprising lipoid acid or a physiologically acceptable salt thereof, binder and, where appropriate, other active substances and/or other physiologically acceptable excipients.

8. A method for oral administration of lipoid acid or of a physiologically acceptable salt thereof, comprising administering a formulation as claimed in claim 1, where appropriate with the addition of other excipients as dosage form.
